(19) United States
(12) Patent Application Publication
(10) Pub. No.: US 2003/0064031 A1
(43) Pub. Date: Apr. 3, 2003

(54) Use of a combination of compounds in a dry powder inhaler

(76) Inventors: Michael John Humphrey, Sandwich (GB); Paul Robert Miller, Sandwich (GB); Michael Trevor Shepherd, Sandwich (GB)

Correspondence Address: PFIZER INC., PATENT DEPARTMENT, MS8260-1611, EASTERN POINT ROAD, GROTON, CT, 06340, US

(21) Appl. No.: 10/236,551

(22) Filed: Sep. 5, 2002

Related U.S. Application Data
(60) Provisional application No. 60/325,709, filed on Sep. 27, 2001.

(30) Foreign Application Priority Data
Sep. 12, 2001 (GB) 0122031.8

(51) Int. Cl. A61L 9/04; A61K 31/4745

(52) U.S. Cl. 424/45; 514/293

(57) ABSTRACT

The present invention relates to an inhaled formulation comprising a combination of a compound selected from a particular class of 5,6-dihydro-9H-pyrazolo[3,4-c]-1,2,4-triazolo[4,3-a]pyridines of the formula (I) and a tiotropium salt or solvate thereof, which is capable of delivering the compound of the formula (I) as fine, solid particles to the lung. The invention also relates to the use of such a formulation in the treatment of certain diseases such as respiratory diseases. By the use of such formulations, it is possible to eliminate the unwanted cough response associated with the use of the compounds of the formula (I) in solution metered dose inhalers, which response can prevent the administration of a therapeutically effective dose and, in the long term, undermine patient compliance.

The present invention relates to an inhaled formulation, comprising a combination of a compound selected from a particular class of 5,6-dihydro-9H-pyrazolo[3,4-c]-1,2,4-triazolo[4,3-a]pyridines of the formula (I) and a tiotropium salt or solvate thereof which is capable of delivering the compound of the formula (I) as fine, solid particles to the lung. The invention also encompasses the use of such a formulation in the treatment of certain diseases such as respiratory diseases.

The compounds that are useful in the invention are the compounds of the formula (I)

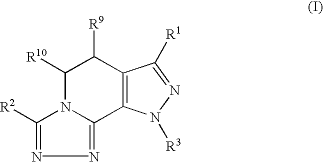

and the pharmaceutically acceptable salts thereof; wherein

R¹ is hydrogen, (C₁-C₆)alkyl, (C₁-C₆)alkoxy, (C₂-C₄)alkenyl, phenyl, dimethylamino, (C₃-C₆)cycloalkyl, (C₃-C₆)cycloalkyl(C₁-C₃)alkyl or (C₁-C₆)acyl wherein the alkyl, phenyl or alkenyl groups may be substituted with up to two hydroxy, (C₁-C₃)alkyl, or trifluoromethyl groups, or up to three halogens;

R² and R³ are each independently selected from the group consisting of hydrogen, (C₁-C₁₄)alkyl, (C₁-C₇)alkoxy(C₁-C₇)alkyl, (C₂-C₁₄)alkenyl, (C₃-C₇)cycloalkyl, (C₃-C₇)cycloalkyl(C₁-C₂)alkyl, a saturated or unsaturated (C₄-C₇)heterocyclic(CH₂)ₙ group wherein n is 0, 1 or 2, containing as the heteroatom one or two of the group consisting of oxygen, sulphur, sulphonyl, nitrogen and NR⁴ wherein R⁴ is hydrogen or (C₁-C₄)alkyl; or a group of the formula

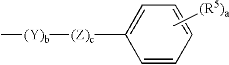

wherein a is an integer from 1 to 5; b and c are 0 or 1; R⁵ is hydrogen, hydroxy, (C₁-C₅)alkyl, (C₂-C₅)alkenyl, (C₁-C₅) alkoxy, (C₃-C₆)cycloalkoxy, halogen, trifluoromethyl, CO₂R⁶, CONR⁶R⁷, NR⁶R⁷, NO₂ or SO₂NR⁶R⁷ wherein R⁶ and R⁷ are each independently hydrogen or (C₁-C₄)alkyl; wherein Z is oxygen, sulphur, SO₂, CO or NR⁸ wherein R⁸ is hydrogen or (C₁-C₄)alkyl; and Y is (C₁-C₅)alkylene or (C₂-C₆)alkenyl optionally substituted with up to two (C₁-C₇)alkyl or

(C₃-C₇)cycloalkyl groups; wherein each of the alkyl, alkenyl, cycloalkyl, alkoxyalkyl or heterocyclic groups may be substituted with one to fourteen of the group consisting of (C₁-C₂)alkyl, trifluoromethyl or halogen; and

R⁹ and R¹⁰ are each independently selected from the group consisting of hydrogen, (C₁-C₆)alkyl, (C₁-C₆)alkoxy, (C₆-C₁₀)aryl and (C₆-C₁₀)aryloxy.

These compounds, which are selective PDE4 inhibitors, are described in International Patent Application WO-A-96/39408. Conditions which may be treated by inhalation of the tricyclic 5,6-dihydro-9H-pyrazolo[3,4-c]-1,2,4-triazolo[4,3-a]pyridines described therein include respiratory diseases such as asthma and chronic obstructive airways disease (COAD, also known as chronic obstructive pulmonary disease (COPD)).

The aforementioned application refers to the optimum therapeutic dose for the compounds described therein as generally in the range of from 0.1 to 400 mg daily for an average adult patient. It is indicated that a dosage for inhaler administration is generally formulated as a 0.1 to 1% (w/v) solution. Although not stated, a typical dosage form for the administration of such a solution would be a metered dose inhaler (MDI).

On the basis of multiple dose patient studies using a solution MDI (administered via a ‘spacer’) to administer small amounts of said compounds at frequent intervals throughout the day, it has been calculated that a daily inhaled dose of up to 3 mg of active compound would be efficacious in the treatment of both asthma and COAD. However, attempts to administer such a quantity by solution MDI using a more reasonable number of doses, typically not more than four per day, invariably produced an immediate cough response in most subjects. Cough severity varied but during the course of the treatment some asthma patients developed worsening of symptoms which was associated with more severe cough responses. Cough responses can prevent the drug being taken on board in a quantity sufficient for the desired therapeutic effect and, perhaps most importantly, have serious consequences for patient compliance.

It has been surprisingly found that when the active compound is administered in the form of fine, solid particles, specifically using a dry powder inhaler, subjects manifest little or no cough response at doses which caused cough with the solution MDI. Subjects are able to accept the full therapeutic dose of active compound or a significant proportion thereof in a reasonable, i.e. patient-compliant, number of doses (typically not more than four per day). This is unexpected since the cough response would normally be associated with the compounds per se and a powder or suspension formulation is potentially irritant.

The present invention provides an inhaled formulation comprising a compound of the formula (I), or a pharmaceutically acceptable salt thereof, as defined above, and a tiotropium salt or solvate thereof, characterised in that the formulation is capable of delivering the compound of the formula (I) as fine, solid particles to the lung.

Further, the present invention provides the use of such an inhaled formulation in the manufacture of a medicament for the treatment of a disease treatable by the inhibition of PDE4, particularly a respiratory disease such as asthma or chronic obstructive pulmonary disease.

Further, the present invention provides a method of treatment of a disease treatable by the inhibition of PDE4, particularly a respiratory disease such as asthma or chronic obstructive pulmonary disease, comprising the administration of such an inhaled formulation to a mammal.

Preferred compounds of the formula (I) for use in the invention have an aqueous solubility at physiological pH of less than 0.15 mg/ml. Compounds having an aqueous solubility of less than 0.05 mg/ml are especially preferred. For the purposes of the invention, “physiological pH” is defined as a pH of from 6.0 to 8.0. Solubility may be measured by diluting a weighed amount of test compound with a suitable pH buffer, shaking the mixture for 24 hours, filtering the mixture and measuring the saturated solubility of the filtrate using LC-MS (liquid chromatography-mass spectrometry).

Preferred compounds of the formula (I) include those wherein each of the alkyl, alkenyl, cycloalkyl, alkoxyalkyl and heterocyclic groups may be substituted with 1 to 5 of the group consisting of (C₁-C₂)alkyl, trifluoromethyl and hydrogen.

Preferably, R¹ is methyl, ethyl or isopropyl.

Preferably, R³ is (C₁-C₆)alkyl, (C₂-C₆)alkenyl, (C₃-C₇)cycloalkyl, (C₃-C₇)cycloalkyl(C₁-C₂)alkyl or phenyl optionally substituted with 1 or 2 of the group consisting of hydrogen, hydroxy, (C₁-C₅)alkyl, (C₂-C₅)alkenyl, (C₁-C₅)alkoxy, halogen, trifluoromethyl, CO₂R⁶, CONR⁶R⁷, NR⁶R⁷, NO₂ or SO₂NR⁶R⁷ wherein R⁶ and R⁷ are each independently hydrogen or (C₁-C₄)alkyl.

Preferred individual compounds of the formula (I) are:

9-cyclopentyl-5,6-dihydro-7-ethyl-3-phenyl-9H-pyrazolo[3,4-c]-1,2,4-triazolo[4,3-α]pyridine;

9-cyclopenyl-5,6-dihydro-7-ethyl-3-(furan-2-yl)-9H-pyrazolo[3,4-c]-1,2,4-triazolo[4,3-α]pyridine;

9-cyclopentyl-5,6-dihydro-7-ethyl-3-(2-pyridyl)-9H-pyrazolo[3,4-c]-1,2,4-triazolo[4,3-α]pyridine;

9-cyclopentyl-5,6-dihydro-7-ethyl-3-(4-pyridyl)-9H-pyrazolo[3,4-c]-1,2,4-triazolo[4,3-α]pyridine;

9-cyclopentyl-5,6-dihydro-7-ethyl-3-(3-thienyl)-9H-pyrazolo[3,4-c]-1,2,4-triazolo[4,3-α]pyridine;

3-benzyl-9-cyclopentyl-5,6-dihydro-7-ethyl-9H-pyrazolo[3,4-c]-1,2,4-triazolo[4,3-α]pyridine;

9-cyclopentyl-5,6-dihydro-7-ethyl-3-propyl-9H-pyrazolo[3,4-c]-1,2,4-triazolo[4,3-α]pyridine;

3,9-dicyclopentyl-5,6-dihydro-7-ethyl-9H-pyrazolo[3,4-c]-1,2,4-triazolo[4,3-α]pyridine;

9-cyclopentyl-5,6-dihydro-7-ethyl-3-(1-methylcyclohex-1-yl)-9H-pyrazolo[3,4-c]-1,2,4-triazolo[4,3-α]pyridine;

3-(tert-butyl)-9-cyclopentyl-5,6-dihydro-7-ethyl-9H-pyrazolo[3,4-c]-1,2,4-triazolo[4,3-α]pyridine;

9-cyclopentyl-5,6-dihydro-7-ethyl-3-(2-methylphenyl)-9H-pyrazolo[3,4-c]-1,2,4-triazolo[4,3-α]pyridine;

9-cyclopentyl-5,6-dihydro-7-ethyl-3-(2-methoxyphenyl)-9H-pyrazolo[3,4-c]-1,2,4-triazolo[4,3-α]pyridine;

9-cyclopentyl-5,6-dihydro-7-ethyl-3-(thien-2-yl)-9H-pyrazolo[3,4]-c[1,2,4-triazolo[4,3-α]pyridine;

3-(2-chlorophenyl)-9-cyclopentyl-5,6-dihydro-7-ethyl-9H-pyrazolo[3,4-c]-1,2,4-triazolo[4,3-α]pyridine;

9-cyclopentyl-5,6-dihydro-7-ethyl-3-(2-iodophenyl)-9H-pyrazolo[3,4-c]-1,2,4-triazolo[4,3-α]pyridine;

9-cyclopentyl-5,6-dihydro-7-ethyl-3-(2-trifluoromethylphenyl)-9H-pyrazolo[3,4-c]-1,2,4-triazolo[4,3-α]pyridine; and

5,6-dihydro-7-ethyl-9-(4-fluorophenyl)-3-(1 -methylcyclohex-1 -yl)-9H-pyrazolo[3,4-c]-1,2,4-triazolo[4,3-α]pyridine;

and the pharmaceutically acceptable salts thereof.

Particularly preferred compounds of the formula (I) are 3-(tert-butyl)-9-cyclopentyl-5,6-dihydro-7-ethyl-9H-pyrazolo[3,4-c]-1,2,4-triazolo[4,3-a]pyridine and 9-cyclopentyl-5,6-dihydro-7-ethyl-3-(thien-2-yl)-9H-pyrazolo[3,4-c]1,2,4-triazolo[4,3-a]pyridine, and the pharmaceutically acceptable salts thereof.

Most preferred are 9-cyclopentyl-5,6-dihydro-7-ethyl-3-(thien-2-yl)-9H-pyrazolo[3,4-c]1,2,4-triazolo[4,3-a]pyridine, and the pharmaceutically acceptable salts thereof, especially the free base.

For the purposes of the present invention, ‘fine’, solid drug particles may be taken to be those which are less than 20 micrometers in diameter. Preferably, the powdered drug used will have a particle size range wherein 90% of particles are less than 10 micrometers in diameter and 50% of particles are less than 5 micrometers in diameter. Even more preferably, the powdered drug used will have a particle size range wherein 90% of particles are less than 6 micrometers in diameter and 50% of particles are less than 3 micrometers in diameter. Most preferably, the powdered drug used will have a particle size range wherein 95% of particles are less than 5 micrometers in diameter and 50% of particles are less than 2.5 micrometers in diameter.

A suitable particle size distribution may be obtained by micronising (milling) the bulk drug substance or by particle engineering. Examples of particle engineering are super critical fluid crystallisation and the preparation of microspheres (e.g. by spray drying).

Devices which are capable of delivering fine, solid particles produced by the techniques outlined above to the lung of a patient include dry powder inhalers, suspension metered dose inhalers, suspension nebulisers and suspension atomisers. Dry powder inhalers are preferred. Suitable dry powder inhalers for use in the invention include capsule devices such as Spinhaler (trade mark), Rotahaler (trade mark), Handihaler (trade mark), Aerohaler (trade mark), Eclipse (trade mark), Turbospin (trade mark) and the Flowcaps (trade mark) inhaler. Other suitable dry powder inhalers for use in the invention include multidose inhalers such as Accuhaler (trade mark), Turbuhaler (trade mark), Ultrahaler (trade mark), Diskhaler (trade mark), Novoliser (trade mark), Easyhaler (trade mark), Taifun (trade mark), Clickhaler (trade mark), Twisthaler (trade mark) and Aspirair (trade mark).

The compounds of the formula (I) and tiotropium can be administered alone but will generally be administered in admixture with a suitable pharmaceutical excipient, diluent or carrier selected with regard to the chosen means of inhalation and standard pharmaceutical practice.

In the case of an aerosol suspension spray presentation from a pressurised container, pump, spray, atomiser (e.g. an atomiser using electrohydrodynamics to produce a fine mist) or nebuliser a suitable propellant may be used such as e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide, a further perfluorinated hydrocarbon such as Perflubron (trade mark) or other suitable gas. In the case of a pressurised aerosol, the dosage unit may be determined by providing a valve to deliver a metered amount. The drug will be dispersed in a suitable agent such as water or aqueous ethanol. A lubricant such as sorbitan trioleate may also be included.

Capsules, blisters and cartridges (made, for example, from gelatin or HPMC) for use in an inhaler may be formulated to contain a powder mix of a compound of the formula (I) and tiotropium, a suitable powder base such as lactose or starch and a performance modifier such as I-leucine, mannitol, trehalose or magnesium stearate. For the purposes of the present invention, a preferred dry powder formulation consists of a dry powder blend of the compound of the formula (I), or salt thereof, tiotropium, and lactose (preferably as lactose monohydrate). The lactose should be of sufficiently fine grade that 90% of the lactose particles are less than 1000 micrometers in diameter and 50% of the lactose particles are less than 500 micrometers in diameter. Preferably, 90% of the lactose particles are less than 300 micrometers in diameter and 50% of the lactose particles are less than 100 micrometers in diameter. Most preferably, 90% of the lactose particles are less than from 100 to 200 micrometers in diameter, 50% of the lactose particles are less than from 40 to 70 micrometers in diameter and 10% of the lactose particles are less than 10 micrometers in diameter. Drug loading may vary from 0.1 to 100% w/w of the dry powder blend and is preferably from 5 to 100% w/w, most preferably from 5-40% w/w.

Aerosol or dry powder formulations are preferably arranged so that each metered dose or “puff” contains from 1 to 10000 μg of a compound of the formula (I) for delivery to the patient. The overall daily dose with an aerosol will be in the range of from 1 μg to 20 mg which may be administered in a single dose or, more usually, in divided doses throughout the day.

A further method of delivering fine, solid particles of drug to the lung is use of microspheres comprising poly(D,L-lactic-co-glycolic acid) wherein such microspheres are generated in situ after delivery from a solution metered dose inhaler.

The fine, solid particles of drug to be delivered according to the invention may optionally be delivered in the form of liposomes to modify their release characteristics.

It is to be appreciated that all references herein to treatment include curative, palliative and prophylactic treatment.

### EXAMPLES

In each of Examples 1 to 3, the lactose monohydrate particle size distribution was 90% less than 190 micrometers in diameter, 50% less than 55 micrometers in diameter and 10% less than 6 micrometers in diameter and the drug particle size distribution was 90% less than 5.8 micrometers in diameter, 50% less than 2.9 micrometers in diameter and 10% less than 1.0 micrometers in diameter.

### Example 1

### Dry Powder Inhaler Capsule (0.5 mg)

A mixture of 9-cyclopentyl-5,6-dihydro-7-ethyl-3-(thien-2-yl)-9H-pyrazolo[3,4-c]-1,2,4-triazolo[4,3-a]pyridine (0.5 mg, micronised by spiral air-jet milling) and lactose monohydrate (9.5 mg, Pharmatose 150M (DMV) Ph.Eur) were blended by hand and filled into a size 3 opaque white capsule shell (supplied by Capsugel, product code 1505).

### Example 2

### Dry Powder Inhaler Capsule (1 mg)

A mixture of 9-cyclopentyl-5,6-dihydro-7-ethyl-3-(thien-2-yl)-9H-pyrazolo[3,4-c]-1,2,4-triazolo[4,3-a]pyridine (1.0 mg, micronised by spiral air-jet milling) and lactose monohydrate (19 mg, Pharmatose 150M (DMV) Ph.Eur) were blended by hand and filled into a size 3 opaque white capsule shell (supplied by Capsugel, product code 1505).

### Example 3

### Dry Powder Inhaler Capsule (2 mg)

A mixture of 9-cyclopentyl-5,6-dihydro-7-ethyl-3-(thien-2-yl)-9H-pyrazolo[3,4-c]-1,2,4-triazolo[4,3-a]pyridine (2.0 mg, micronised by spiral air-jet milling) and lactose monohydrate (38 mg, Pharmatose 150M (DMV) Ph.Eur) were blended by hand and filled into a size 3 opaque white capsule shell (supplied by Capsugel, product code 1505).

### Example 4

### Dry Powder Inhaler

The capsules manufactured in accordance with Examples 1 to 3 were loaded into a monodose inhaler (supplied by Plastiape SpA) for administration to human subjects.

### Example 5

### Clinical Data

The formulations of Examples 1 to 3 were tested for tolerance and safety in a clinical trial using healthy volunteers. Volunteers were dosed using a dry powder inhaler, as described in Example 4, with capsules containing the 0.5 mg, 1 mg and 2 mg doses of Examples 1, 2 and 3, respectively, or with placebo capsules containing only lactose. A dose escalation was used and any coughs were assessed as to their number, severity, duration and quality. Some of the results are shown in Table 1.

TABLE 1
Cough toleration

| | Percentage of subjects coughing | |
|---|---|---|
| | in the first 5 minutes | |
| | following dosing | |
| Dose | Placebo (dry powder) | Active (dry powder) |
| 1 × 0.5 mg | — | 0 (0/9) |
| 2 × 0.5 mg | — | 11 (1/9) |
| 1 × 1 mg | 0 (0/6) | 22 (2/9) |
| 2 × 1 mg | 0 (0/6) | 22 (2/9) |
| 1 × 2 mg | 0 (0/3) | 33 (3/9) |
| 2 × 2 mg | 0 (0/3) | 11 (1/9) |
| 3 × 2 mg | 0 (0/3) | 29 (2/7) |

When a dose equivalent to the 1×0.5 mg dry powder dose is administered via a solution MDI, approximately 80-100% of subjects experience cough in the first 5 minutes following dosing. Not only is the percentage of cough greatly reduced by use of the dry powder inhaler as compared with the solution metered dose inhaler but the severity of those coughs is also greatly reduced. Furthermore, with the dry powder inhaler the incidence of cough remains acceptable at doses in the therapeutic range.

1. An inhaled formulation comprising a combination of a compound of the formula (I) 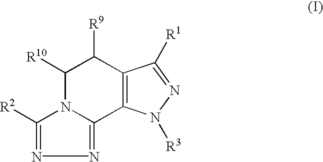
or a pharmaceutically acceptable salt thereof; wherein
R¹ is hydrogen, (C₁-C₆)alkyl, (C₁-C₆)alkoxy, (C₂-C₄)alkenyl, phenyl, dimethylamino, (C₃-C₆)cycloalkyl, (C₃-C₆)cycloalkyl(C₁-C₃)alkyl or (C₁-C₆)acyl wherein the alkyl, phenyl or alkenyl groups may be substituted with up to two hydroxy, (C₁-C₃)alkyl, or trifluoromethyl groups, or up to three halogens;
R² and R³ are each independently selected from the group consisting of hydrogen, (C₁-C₁₄)alkyl, (C₁-C₇)alkoxy(C₁-C₇)alkyl, (C₂-C₁₄)alkenyl, (C₃-C₇)cycloalkyl, (C₃-C₇)cycloalkyl(C₁-C₂)alkyl, a saturated or unsaturated (C₄-C₇)heterocyclic(CH₂)ₙ group wherein n is 0, 1 or 2, containing as the heteroatom one or two of the group consisting of oxygen, sulphur, sulphonyl, nitrogen and NR⁴ wherein R⁴ is hydrogen or (C₁-C₄)alkyl; or a group of the formula 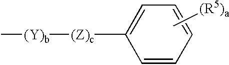
wherein a is an integer from 1 to 5; b and c are 0 or 1; R⁵ is hydrogen, hydroxy, (C₁-C₅)alkyl, (C₂-C₅)alkenyl, (C₁-C₅) alkoxy, (C₃-C₆)cycloalkoxy, halogen, trifluoromethyl, CO₂R⁶, CONR⁶R⁷, NR⁶R⁷, NO₂ or SO₂NR⁶R⁷ wherein R⁶ and R⁷ are each independently hydrogen or (C₁-C₄)alkyl; wherein Z is oxygen, sulphur, SO₂, CO or NR⁸ wherein R⁸ is hydrogen or (C₁-C₄)alkyl; and Y is (C₁-C₅)alkylene or (C₂-C₆)alkenyl optionally substituted with up to two (C₁-C₇)alkyl or (C₃-C₇)cycloalkyl groups; wherein each of the alkyl, alkenyl, cycloalkyl, alkoxyalkyl or heterocyclic groups may be substituted with one to fourteen of the group consisting of (C₁-C₂)alkyl, trifluoromethyl or halogen; and
R⁹ and R¹⁰ are each independently selected from the group consisting of hydrogen, (C₁-C₆)alkyl, (C₁-C₆)alkoxy, (C₆-C₁₀)aryl and (C₆-C₁₀)aryloxy;
and a tiotropium salt or solvate thereof characterised in that the formulation is capable of delivering the compound of the formula (I) as fine, solid particles to the lung.

2. An inhaled formulation according to claim 1 wherein each of the alkyl, alkenyl, cycloalkyl, alkoxyalkyl and heterocyclic groups may be substituted with 1 to 5 of the group consisting of (C₁-C₂)alkyl, trifluoromethyl and hydrogen.

3. An inhaled formulation according to claim 1 wherein R³ is methyl, ethyl or isopropyl.

4. An inhaled formulation according to claim 1 wherein R³ is (C₁-C₆)alkyl, (C₂-C₆)alkenyl, (C₃-C₇)cycloalkyl, (C₃-C₇)cycloalkyl(C₁-C₂)alkyl or phenyl optionally substituted with 1 or 2 of the group consisting of hydrogen, hydroxy, (C₁-C₅)alkyl, (C₂-C₅)alkenyl, (C₁-C₅)alkoxy, halogen, trifluoromethyl, CO₂R⁶, CONR⁶R⁷, NR⁶R⁷, NO₂ or SO₂NR⁶R⁷ wherein R⁶ and R⁷ are each independently hydrogen or (C₁-C₄)alkyl.

5. An inhaled formulation according to claim 1 wherein the compound of the formula (I) is selected from:
9-cyclopentyl-5,6-dihydro-7-ethyl-3-phenyl-9H-pyrazolo[3,4-c]-1,2,4-triazolo[4,3-α]pyridine;
9-cyclopenyl-5,6-dihydro-7-ethyl-3-(furan-2-yl)-9H-pyrazolo[3,4-c]-1,2,4-triazolo[4,3-α]pyridine;
9-cyclopentyl-5,6-dihydro-7-ethyl-3-(2-pyridyl)-9H-pyrazolo[3,4-c]-1,2,4-triazolo[4,3-α]pyridine;
9-cyclopentyl-5,6-dihydro-7-ethyl-3-(4-pyridyl)-9H-pyrazolo[3,4-c]-1,2,4-triazolo[4,3-α]pyridine;
9-cyclopentyl-5,6-dihydro-7-ethyl-3-(3-thienyl)-9H-pyrazolo[3,4-c]-1,2,4-triazolo[4,3-α]pyridine;
3-benzyl-9-cyclopentyl-5,6-dihydro-7-ethyl-9H-pyrazolo[3,4-c]-1,2,4-triazolo[4,3-α]pyridine;
9-cyclopentyl-5,6-dihydro-7-ethyl-3-propyl-9H-pyrazolo[3,4-c]-1,2,4-triazolo[4,3-α]pyridine;
3,9-dicyclopentyl-5,6-dihydro-7-ethyl-9H-pyrazolo[3,4-c]-1,2,4-triazolo[4,3-α]pyridine;
9-cyclopentyl-5,6-dihydro-7-ethyl-3-(1-methylcyclohex-1-yl)-9H-pyrazolo[3,4-c]-1,2,4-triazolo[4,3-α]pyridine;
3-(tert-butyl)-9-cyclopentyl-5,6-dihydro-7-ethyl-9H-pyrazolo[3,4-c]-1,2,4-triazolo[4,3-α]pyridine;
9-cyclopentyl-5,6-dihydro-7-ethyl-3-(2-methylphenyl)-9H-pyrazolo[3,4-c]-1,2,4-triazolo[4,3-α]pyridine;
9-cyclopentyl-5,6-dihydro-7-ethyl-3-(2-methoxyphenyl)-9H-pyrazolo[3,4-c]-1,2,4-triazolo[4,3-α]pyridine;
9-cyclopentyl-5,6-dihydro-7-ethyl-3-(thien-2-yl)-9H-pyrazolo[3,4-c]-1,2,4-triazolo[4,3-α]pyridine;
3-(2-chlorophenyl)-9-cyclopentyl-5,6-dihydro-7-ethyl-9H-pyrazolo[3,4-c]-1,2,4-triazolo[4,3-α]pyridine;
9-cyclopentyl-5,6-dihydro-7-ethyl-3-(2-iodophenyl)-9H-pyrazolo[3,4-c]-1,2,4-triazolo[4,3-α]pyridine;
9-cyclopentyl-5,6-dihydro-7-ethyl-3-(2-trifluoromethylphenyl)-9H-pyrazolo[3,4-c]-1,2,4-triazolo[4,3-α]pyridine; and
5,6-dihydro-7-ethyl-9-(4-fluorophenyl)-3-(1-methylcyclohex-1-yl)-9H-pyrazolo[3,4-c]-1,2,4-triazolo[4,3-a]pyridine;
and the pharmaceutically acceptable salts thereof.

6. An inhaled formulation according to claim 1 wherein the compound of the formula (I) is 3-(tert-butyl)-9-cyclopentyl-5,6-dihydro-7-ethyl-9H-pyrazolo[3,4-c]-1,2,4-triazolo[4,3-a]pyridine.

7. An inhaled formulation according to claim 1 wherein the compound of the formula (I) is 9-cyclopentyl-5,6-dihydro-7-ethyl-3-(thien-2-yl)-9H-pyrazolo[3,4-c]-1,2,4-triazolo[4,3-a]pyridine.

8. An inhaled formulation according to claim 1 wherein the compound of the formula (I) has an aqueous solubility at physiological pH of less than 0.15 mg/ml.

9. An inhaled formulation according to claim 8 wherein the compound of the formula (I) has an aqueous solubility at physiological pH of less than 0.05 mg/ml.

10. An inhaled formulation according to claim 1 wherein the fine, solid drug particles have a size distribution of 90% less than 10 micrometers in diameter and 50% less than 5 micrometers in diameter.

11. An inhaled formulation according to claim 10 wherein the fine, solid drug particles have a size distribution of 90% less than 6 micrometers in diameter and 50% less than 3 micrometers in diameter.

12. An inhaled formulation according to claim 1 wherein the fine, solid particles are delivered to the lung by a dry powder inhaler.

13. An inhaled formulation according to claim 12 wherein the dry powder blend comprises lactose, preferably in its monohydrated form.

14. An inhaled formulation according to claim 1 wherein the fine, solid particles are delivered by a suspension metered dose inhaler, a suspension atomiser or a suspension nebuliser.

15. An inhaled formulation according to claim 1 wherein the fine, solid particles consist of microspheres, said microspheres comprising poly(D,L-lactic-co-glycolic acid).

16. An inhaled formulation according to claim 1 for use as a medicament.

17. The use of an inhaled formulation according to any one of claim 1 in the manufacture of a medicament for the treatment of a disease treatable by the inhibition of PDE4.

18. The use according to claim 17, wherein the disease is a respiratory disease.

19. The use according to claim 18 wherein the respiratory disease is asthma or chronic obstructive pulmonary disease.

20. A method of treatment of a disease treatable by the inhibition of PDE4 comprising the administration of an inhaled formulation according to claim 1 to a mammal.

21. A method of treatment according to claim 20 wherein the disease is a respiratory disease.

22. A method of treatment according to claim 21 wherein the respiratory disease is asthma or chronic obstructive pulmonary disease.
